(19) United States
(12) Patent Application Publication
(10) Pub. No.: US 2005/0132448 A1
(43) Pub. Date: Jun. 16, 2005

(54) Assay for beta cell toxic macrolides

(76) Inventors: Mark A Myers, Clayton, Victoria (AU); Ian R. Mackay, Clayton, Victoria (AU); Paul Z. Zimmet, Caulfield, Victoria (AU)

Correspondence Address: William T Christiansen;Seed Intellectual Property Law Group, Suite 6300, 701 5th Avenue, Seattle, WA, 98104-7092, US

(21) Appl. No.: 10/479,127

(22) PCT Filed: May 22, 2002
(86) PCT No.: PCT/AU02/00643

(30) Foreign Application Priority Data
May 31, 2001 (AU) PR 5371

(51) Int. Cl. A01H 1/00; C12N 15/82; C12Q 1/00; A61K 31/33

(52) U.S. Cl. 800/288; 435/4; 514/28; 514/183

(57) ABSTRACT

The present invention relates to a method of assessing the propensity of a plant, if consumed, to contribute to the onset and/or progression of diabetes in a mammal and, more particularly, to a method of assessing the propensity of a tuberous vegetable, if consumed, to contribute to the onset and/or progression of diabetes in a mammal. The method of the present invention is useful, inter alia, for identifying mammals at risk of developing diabetes based on dietary intake. The present invention is further directed to methods of assessing the risk status of an individual for development of diabetes. The present invention still further provides methods for the prophylactic and/or therapeutic treatment of diabetes.

### FIELD OF THE INVENTION

The present invention relates to a method of assessing the propensity of a plant, if consumed, to contribute to the onset and/or progression of diabetes in a mammal and, more particularly, to a method of assessing the propensity of a tuberous vegetable, if consumed, to contribute to the onset and/or progression of diabetes in a mamma. The method of the present invention is useful, inter alia, for identifying mammals at risk of developing diabetes based on dietary intake. The present invention is further directed to methods of assessing the risk status of an individual for development of diabetes. The present invention still further provides methods for the prophylactic and/or therapeutic treatment of diabetes.

### BACKGROUND OF THE INVENTION

Bibliographic details of the publications referred to by author in this specification are collected at the end of the description.

The reference to any prior art in this specification is not, and should not be taken as, an acknowledgment or any form of suggestion that that prior art forms part of the common general knowledge in Australia.

Diabetes mellitus is characterised by an abnormality of carbohydrate metabolism resulting in elevated glucose levels in both the blood and the urine. The failure of the human body to properly metabolise the glucose results particularly from decreased insulin secretion relative to requirements. Insulin is produced by β-cells in the islets of the pancreas and permits the body to utilise glucose as a source of energy. When this process cannot occur, the body compensates by utilising alternative sources of energy such as stored fats. However, this leads to rapidly rising levels of glucose and the accumulation of ketones in the bloodstream due to the occurrence of extensive fat metabolism. When left untreated, these events lead to a life threatening condition termed “diabetic ketoacidosis”.

Diabetes is broadly classified into two groups termed Type 1 diabetes and Type 2 diabetes. Type 1 diabetes (often referred to as juvenile onset diabetes due to its appearance in childhood or early adolescence) is a debilitating autoimmune condition caused by the selective destruction of insulin producing β-cells in the islets of the pancreas. Its onset is abrupt and occurs typically prior to the age of 20 years. Presently, however, Type 1 diabetes is increasingly presenting in adults. This disease is characterised by lack of β-cell function and no insulin production, and therefore insulin therapy is required. Type 2 diabetes, however, is characterised by a gradual onset in individuals who are generally over the age of 35 years and is often accompanied by obesity and other metabolic disorders. There are frequently no overt symptoms observed. This condition is characterised by abnormal β-cell function.

Insulin secretory defects are evident very early in disease in both Type 1 and Type 2 diabetes, despite their differing aetiology. A particular feature of Type 2 diabetes is an increased secretion of proinsulin and its partial cleavage products (Temple et al., 1989). This may result from the increased insulin requirement imposed by peripheral and hepatic insulin resistance leading to metabolic stress of the β-cells and premature release of proinsulin, although an intrinsic β-cell defect that precedes or acts in concert with insulin resistance has also been proposed (Porte, 1999). Increased proinsulin secretion also occurs in the preclinical stages of Type 1 diabetes (Chaillous et al., 1996; Roder et al., 1994; Lindgren et al., 1991). In a mouse model of Type 1 diabetes, the non-obese diabetic (NOD) mouse there is similarly an altered glucose response of β-cells and hyperinsulinemia before the onset of insulitis and overt diabetes (Amrani et al., 1998). In preclinical stages of Type 1 diabetes in humans and NOD mice, there is no evidence of insulin resistance or hyperglycaemia. Rather, the insulin secretory defect has an autoimmune basis, i.e. is intrinsic, with an inflammatory background associated with the induction of nitric oxide synthesis upon exposure of the β-Cells to cytokines (Arnush et al., 1998; Corbett et al., 1992; Hostens et al., 1999; Rabinovitch, 1998; Sjoholm, 1998).

While the insulin secretion defects and hyperproinsulinemia associated with Type 1 and Type 2 diabetes appear to have different provenance, certain of the causative biochemical mechanisms may be similar. The lumen of the immature secretory granules from which insulin is released must be acidified, which allows for efficient cleavage of newly synthesised proinsulin molecules by prohormone convertases (PCs) to yield proinsulin and C-peptide (Orci et al., 1994; Paquet et al., 1996). Granule acidification is an essential step, as neutralisation of secretory granule activity results in incomplete proinsulin cleavage (Orci et al., 1994) and leakage of proinsulin into the constitutive secretory pathway (Kuliawat and Arvan, 1994). Acidification is mediated by the translocation of protons into vesicular compartments by the vacuolar ATPase (vATPase) (Forgac, 1999). Proton pumping by the vATPase is entirely dependent on ATP and so will be sensitive to conditions that reduce cellular ATP levels, such as metabolic stress imposed on β-cells by insulin resistance and hyperglycaemia in Type 2 diabetes. vATPase activity is also sensitive to nitric oxide (Tojo et al., 1994; Forgac, 1999; Swallow et al., 1991). Consequently, induction of nitric oxide synthesis in β-cells upon exposure to cytokines, as occurs in the insulitis stages of Type 1 diabetes, would reduce vATPase activity. Thus, similar biochemical processes may mediate the hyperproinsulinemia seen in both Type 1 and Type 2 diabetes, despite the different causations leading to β-cell dysfunction in the two diseases.

Accordingly, there is an ongoing need to identify the factors which induce or otherwise modulate the biochemical dysregulation associated with diabetes. In work leading up to the present invention, the inventors have surprisingly determined that species of Streptomyces which can infest tuberous vegetables produce certain macrolides which adversely affect β-cell functional activity. Thus consumption of the infected vegetable can lead to the onset of diabetes. These macrolides are also thought to function by inhibiting vATPase activity and so down-regulating basal insulin secretion but may have a direct toxic effect on the β cell. The identification of this environmental contribution to diabetes has now facilitated the development of methodology directed to assessing the propensity of an edible plant, such as a tuberous vegetable, to contribute to the onset and/or progression of diabetes. There is also now provided methodology for screening individuals to assess levels of macrolide ingestion, as an indicator of exposure to a diabetes causative agent The present invention further facilitates the development of diabetes-related therapeutic and prophylactic protocols based on modulating ingested macrolide functional activity, such as by neutralising said macrolides or modulating dietary intake, in order to decrease macrolide intake in an individual with, or at risk of diabetes.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Accordingly, one aspect of the present invention is directed to a method for assessing the propensity of an ingestible agent to induce, up-regulate or otherwise contribute, subsequently to its ingestion by a mammal, to the onset, and/or progression of diabetes in said mammal, said method comprising screening said agent for the expression of one or more β-cell toxic macrolide or derivatives, variants, mutants or homologues thereof wherein expression of said macrolide is indicative of the propensity of said agent to induce, up-regulate or otherwise contribute to the onset and/or progression of diabetes.

Another aspect of the present invention is directed to a method for assessing the propensity of a plant or propagation material thereof to induce, up-regulate or otherwise contribute, subsequently to its consumption by a mammal, to the onset and/or progression of diabetes in said mammal, said method comprising screening said plant or propagation material thereof for the expression of one or more β-cell toxic macrolides or derivatives, variants, mutants or homologues thereof wherein expression of said macrolide is indicative of the propensity of said plant or propagation material thereof to induce, up-regulate or otherwise contribute to the onset and/or progression of diabetes.

Still another aspect of the present invention more particularly provides a method for assessing the propensity of a tuberous vegetable to induce, up-regulate or otherwise contribute, subsequently to its consumption by a mammal, to the onset and/or progression of diabetes in said mammal, said method comprising screening said tuberous vegetable for the expression of one or more β-cell toxic macrolides or derivatives, variants, mutants or homologues thereof wherein expression of said macrolide is indicative of the propensity of said tuberous vegetable to induce, up-regulate or otherwise contribute to the onset and/or progression of diabetes.

In yet another aspect there is provided a method for assessing the propensity of a potato to induce, up-regulate or otherwise contribute, subsequently to its consumption by a mammal, to the onset and/or progression of diabetes in said mammal, said method comprising screening said potato for the expression of one or more β-cell toxic macrolides or derivatives, variants, mutants or homologues thereof wherein expression of said macrolide is indicative of the propensity of said potato to induce, up-regulate or otherwise contribute to the onset and/or progression of diabetes.

Still yet another aspect there is provided a method for assessing the propensity of a beet to induce, up-regulate or otherwise contribute, subsequently to its consumption by a mammal, to the onset and/or progression of diabetes in said mammal, said method comprising screening said beet for the expression of one or more β-cell toxic macrolides or derivatives, variants, mutants or homologues thereof wherein expression of said macrolide is indicative of the propensity of said beet to induce, up-regulate or otherwise contribute to the onset and/or progression of diabetes.

Yet still another aspect there is provided a method for assessing the propensity of a carrot to induce, up-regulate or otherwise contribute, subsequently to its consumption by a mammal, to the onset and/or progression of diabetes in said mammal, said method comprising screening said carrot for the expression of one or more β-cell toxic macrolides or derivatives, variants, mutants or homologues thereof wherein expression of said macrolide is indicative of the propensity of said carrot to induce, up-regulate or otherwise contribute to the onset and/or progression of diabetes.

In still another aspect there is provided a method for assessing the propensity of a plant or propagation material thereof to induce, up-regulate or otherwise contribute, subsequently to its consumption by a mammal, to the onset and/or progression of diabetes in said mammal, said method comprising screening said plant or propagation material thereof for the expression of one or more bafilomycin macrolides or derivatives, variants, mutants or homologues thereof wherein expression of said bafilomycin macrolide is indicative of the propensity of said plant or propagation material thereof to induce, up-regulate or otherwise contribute to the onset and/or progression of diabetes.

In still yet another aspect there is provided a method for assessing the propensity of a plant or propagation material thereof to induce, up-regulate or otherwise contribute, subsequently to its consumption by a mammal, to the onset and/or progression of diabetes in said mammal, said method comprising screening said plant or propagation material thereof for the expression of one or more concanamycin macrolides or derivatives, variants, mutants or homologues thereof wherein expression of said concanamycin A is indicative of the propensity of said plant or propagation material thereof to induce, up-regulate or otherwise contribute to the onset and/or progression of diabetes.

In yet still another aspect there is provided a method of diagnosing the existence of a diabetes risk factor in a mammal, said method comprising screening said mammal for the expression of one or more β-cell toxic macrolides or derivative, variant, mutant or homologue thereof wherein the expression of said macrolide may be indicative of the risk of onset and/or progression of diabetes in said mammal.

A further aspect of the present invention provides a method of diagnosing the existence of a diabetes risk factor in a human said method comprising screening said human for the expression of one or more bafilomycin macrolides or derivative, variant, mutant or homologue thereof wherein the expression of said bafilomycin may be indicative of the risk of onset and/or progression of diabetes in said human.

Preferably said bafilomycin is bafilomycin A1, A2, B1, B2 and/or C.

In another preferred aspect there is provided a method of diagnosing the existence of a diabetes risk factor in a human said method comprising screening said human for the expression of one or more concanamycin macrolides or derivative, variant, mutant or homologue thereof wherein the expression of said concanamycin A may be indicative of the risk of onset and/or progression of diabetes in said human.

Preferably said concanamycin is concanamycin A, B, C and/or D.

In yet another aspect of the present invention there is provided a diagnostic kit for assaying plants, propagation material thereof, or a biological sample comprising in a compartmental form a first compartment adapted to contain an agent for detecting a β-cell toxic macrolide and a second compartment adapted to contain reagents useful for facilitating the detection by the agent in the first compartment. Further compartments may also be included, for example, to receive a biological sample. The agent may be an antibody or other suitable detection molecule.

Another further aspect of the present invention is directed to a method of preventing, reducing or otherwise ameliorating diabetes in a mammal, said method comprising down-regulating the functional activity β-cell toxic macrolides or derivatives, variants, mutants or homologues thereof, expressed by said mammal.

In yet another further aspect the present invention contemplates a pharmaceutical composition comprising a β-cell toxic macrolide antagonist together with one or more pharmaceutically acceptable carriers and/or diluents. The antagonist is referred to as the active ingredient.

It should be understood that in addition to down-regulating the functional activity of β-cell toxic macrolides which have been ingested by a mammal, the present invention also extends to decreasing the functional activity of β-cell toxic macrolides prior to their ingestion by a mammal.

In yet another aspect there is provided a method of preventing, reducing or otherwise ameliorating diabetes in a mammal, said method comprising reducing consumption by said mammal of plants, or propagation material thereof, which plants express one or more β-cell toxic macrolides or derivatives, variants, mutants or homologues thereof.

### BRIEF DESCRIPTION OF THE DRAWINGS

FIG. 1 is a graphical representation of oral glucose tolerance tests. Controls are derived from 3 measurements at each time point for each of 6 separate mice. Bafilomycin A1 treated data is derived from 1 measurement at each time point from 6 separate mice. There are significant differences using Student's t-test for these observations:
7 day versus all controls at 120 minutes p=0.006
21 day versus all controls at 15 minutes p=0.04
21 day versus all controls at 120 minutes p=0.009

FIG. 2 is a graphical representation of vacuolar ATPase inhibition causing increased islet insulin content of secretory granules in islet β cells. Infra-islet insulin was detected in paraffin embedded sections by indirect immunofluorescence using an anti-pig insulin antibody. Digitised images collected on an epifluorescent microscope were analysed for staining density using MCID image analysis software to provide a semi-quantitative measure of the amount of bound anti-insulin antibody. At least 9 islets were measured for each of 5 control mice and 6 bafilomycin A1-treated mice, and individual values for each islet from all mice in each group were combined for analysis. A measurement was made in each islet, and the background measured in exocrine tissue adjacent to the same islet was subtracted. Units are arbitrary fluorescence units and the data are expressed as the mean and standard deviation.

FIG. 3 is a graphical representation of the amount of insulin released by the mouse islet β-cell line MIN6 after exposure to 10 nM bafilomycin A1 for 3 hours. The culture medium was changed to one containing 0, 4, 16, or 24 mM glucose and inhibitor and left for a further 3 hours. Insulin release was measured by a radioimmunoassay.

FIG. 4. Mice given bafilomycin A1 on 2 separate occasions a week apart were followed for 90 days after treatment. No significant difference in the weight of the inhibitor treated or carrier treated groups was observed. Although random blood glucose (RBG) levels did differ between the groups in that the treated mice showed an increased level of RBG first detectable 50 days after treatment (p<0.05, repeated measures ANOVA), although the RBGs of all mice remained within the normal range over the observation period.

FIG. 5 is an image of the islets from bafilomycin A1 treated mice showing a markedly altered morphology. Paraffin embedded sections of mouse pancreas immunostained for insulin and counterstained with haematoxylin. A represents an untreated control and B represents a treated mouse and shows islet disorganisation exemplified in C by an islet cell cluster associated with a pancreatic duct.

FIG. 6 is a graphical representation of islet size following administration of bafilomycin A1. Reduced islet size is a long-term consequence of vacuolar ATPase inhibition. Pancreas morphology was examined at either 1, 7, 26 or 90 days after administration of bafilomycin A1. Islet size was determined by measuring the areas of the pancreas sections positive for staining with insulin antibody using MCID software.
Controls—223 islets from 12 mice
1 day—81 islets from 3 mice
7 days—125 islets from 3 mice
26 days—90 islets from 4 mice
90 days—81 islets from 4 mice
The black square represents the median, the dotted boxes 25-75^{th} percentiles, bars the 10-90^{th} percentiles, and outliers by dots.

FIG. 7 is a graphical representation of the RP-HPLC Chromatographic profile of an ethyl acetate extract of Streptomyces sp. EF-73 isolated from a potato scab lesion. The column was developed with a linear 30-100% acetonitrile gradient followed by an isocratic 100% acetonitrile step. Peaks were detected by absorption (AU) at 254 nm. Peaks 1, 5 and 6 (*) were able to inhibit acridine orange uptake in the sensitive bioassay indicating the presence of vATPase inhibitors/ionophores in these fractions. Preliminary Mass Spectrometry data of peak 5 revealed a molecular mass of 816 Daltons, characteristic of a Bafilomycin (Bafilomycin B₁), and the retention times of peaks 5 and 6 correspond closely to that of authentic Bafilomycin B₁ (indicated by arrow).

FIG. 8 is a graphical representation of the percentage of diabetes free mice at regular time intervals following BA1 administration.

### DETAILED DESCRIPTION OF THE INVENTION

The present invention is predicated, in part, on the surprising and unexpected determination that macrolides from ubiquitous soil organisms can adversely affect pancreatic islet β-cell functioning and viability and, still further, that orally administered macrolides will adversely affect β-cell functioning. These findings have therefore led to the determination that β-cell toxic macrolides which enter the human diet can contribute to the onset and/or progression of diabetes. Accordingly, these findings have facilitated the development of screening methodology directed to identifying plant material, in particular that destined for human consumption, which would exhibit a propensity to induce diabetes, for example, in genetically susceptible individuals. These findings have further facilitated the development of diagnostic methodology directed to establishing whether an individual, such as a genetically susceptible individual, is ingesting one or more of the subject macrolides and is therefore at risk of, or may have already developed, one or more symptoms of diabetes. Still further, development of prophylactic and/or therapeutic protocols directed to down-regulating the functional activity of an ingested macrolide load of an individual or reducing macrolide ingestion, per se, are herewith facilitated.

Accordingly, one aspect of the present invention is directed to a method for assessing the propensity of an ingestible agent to induce, up-regulate or otherwise contribute, subsequently to its ingestion by a mammal, to the onset, and/or progression of diabetes in said mammal, said method comprising screening said agent for the expression of one or more β-cell toxic macrolide or derivatives, variants, mutants or homologues thereof wherein expression of said macrolide is indicative of the propensity of said agent to induce, up-regulate or otherwise contribute to the onset and/or progression of diabetes.

Reference to “agent” should be understood as a reference to any naturally or non-naturally occurring composition or molecule. In accordance with the present invention, the agent is one which is ingestible. By “ingestible” is meant that the agent is taken into the body of the mammal. This may occur by any route such as orally, intravenously or intradermally. In a preferred embodiment, the agent is a plant which is ingested orally.

The present invention is therefore more particularly directed to a method for assessing the propensity of a plant or propagation material thereof to induce, up-regulate or otherwise contribute, subsequently to its consumption by a mammal, to the onset and/or progression of diabetes in said mammal, said method comprising screening said plant or propagation material thereof for the expression of one or more β-cell toxic macrolides or derivatives, variants, mutants or homologues thereof wherein expression of said macrolide is indicative of the propensity of said plant or propagation material thereof to induce, up-regulate or otherwise contribute to the onset and/or progression of diabetes.

As detailed hereinbefore, the unexpected determination that some macrolides adversely affect β-cell function has facilitated the still more surprising determination of a link between diet and diabetes onset. Specifically, macrolides are expressed by Streptomyces species, which bacteria are known, inter alia, to exist in soil and infest plant material located therein. Streptomyces are a genus of gram positive spore forming bacteria. Without limiting the theory or object of the present invention in any way, they are known to grow slowly in soil and water as branching filamentous mycelium similar to that of fungi. They are a source of many antibiotics including streptomycin, tetracycline, chloramphenicol and macrolides. It should be understood that the Streptomyces species which produce the subject macrolides may co-exist in several different relationships with the plant. For example, the subject Streptomyces may divide and survive as colonies which are located in the soil. Due to the proximal location of these colonies to a part of the plant, such as the root system or propagation materials such as a tuberous vegetable, some of these Streptomyces may be passively transferred to the plant where the subject Streptomyces may continue to maintain viability and metabolise (thereby producing macrolides) or where they may, alternatively, form an active infestation in the form of colony establishment and continued expansion. The subject Streptomyces may be located in any region of the plant, such as on the surface of the plant or within the plant tissue itself. Some actinomycetes are known to exist in endophytic relationships with a plant and to be transferred to new plant crops via the propagation material of the infected parent plant. Further, although many endophytic actinomycetes colonise the root systems, or other tissues located within the soil, of the plant, they are also known to coexist with the plant at other locations such as in the leaves or stems.

The term “expression” should therefore be understood in its broadest sense to refer to any form of association of a β-cell toxic macrolide with the subject plant such as, but not limited to, passive adsorption or absorption of the macrolide, per se, or Streptomyces bacterium directly from the soil onto the surface of the plant or production of macrolide by Streptomyces which have colonised the subject plant either on or close to the surface of the plant or which exist in an endophytic relationship with the plant. It should also be understood that the plant, or propagation material thereof, “expresses” a macrolide provided that the macrolide is associated with at least one region of the plant. It is not necessary that the macrolide be detectable throughout the entire plant. Accordingly, as will hereinafter be described in more detail, although a preferred embodiment of the present invention is the screening of tuberous vegetables, such as potatoes, for Streptomyces infection, the present invention should nevertheless be understood to extend to the screening of any type of plant which may be the subject of consumption. Further, any part of the subject plant may be the subject of screening such as those regions which may be the subject of consumption or any other suitable region, the screening results of which would be indicative of the infection status of that part of the plant which would be proposed to be the subject of a consumption.

Reference to a “plant” should therefore be understood as a reference to any naturally or non-naturally occurring plant. For example, flowering crops, cereal crops (eg. wheat and barley) and horticultural crops (eg. tomatoes, beet, carrot and potatoes). By “non-naturally” is meant that the subject plant has undergone some form of manipulation or modification prior to it being screened in accordance with the method of the present invention. Examples of manipulation include, but are not limited to, genetic modification of a plant or treatment of a seedling or propagation material with an extraneous, proteinaceous or non-proteinaceous molecule. Genetic modification may be performed, for example, to improve productivity characteristics or to introduce one or more biocontrol characteristics. The non-naturally occurring plant may be derived from any source. For example, to the extent that the non-naturally occurring plant is one which is genetically modified, the plant may be one which has itself undergone genetic modification or it may have been cultivated from a seed which has undergone genetic modification. Alternatively, the plant may be derived from a seed which itself was derived from a genetically modified plant. Preferably the plant is a tuberous vegetable plant and even more particularly a potato plant, beet plant or carrot plant.

Reference to “propagation material” should be understood as a reference to any type of cellular material from which a plant would germinate or otherwise arise. Examples of propagating material include, but are not limited to, a tuber, a seed, cutting or cell suspension. The propagating material may take any suitable form. For example, it may have been freshly harvested or it may be derived from a stock sample, such as a tuber sample or a frozen stock of cells which have been stored prior to screening. Preferably said propagation material is a tuberous vegetable and even more particularly a potato, beet or carrot.

Accordingly, the present invention still more particularly provides a method for assessing the propensity of a tuberous vegetable to induce, up-regulate or otherwise contribute, subsequently to its consumption by a mammal, to the onset and/or progression of diabetes in said mammal, said method comprising screening said tuberous vegetable for the expression of one or more β-cell toxic macrolides or derivatives, variants, mutants or homologues thereof wherein expression of said macrolide is indicative of the propensity of said tuberous vegetable to induce, up-regulate or otherwise contribute to the onset and/or progression of diabetes.

In a preferred embodiment, there is provided a method for assessing the propensity of a potato to induce, up-regulate or otherwise contribute, subsequently to its consumption by a mammal, to the onset and/or progression of diabetes in said mammal, said method comprising screening said potato for the expression of one or more β-cell toxic macrolides or derivatives, variants, mutants or homologues thereof wherein expression of said macrolide is indicative of the propensity of said potato to induce, up-regulate or otherwise contribute to the onset and/or progression of diabetes.

In another preferred embodiment, there is provided a method for assessing the propensity of a beet to induce, up-regulate or otherwise contribute, subsequently to its consumption by a mammal, to the onset and/or progression of diabetes in said mammal, said method comprising screening said beet for the expression of one or more β-cell toxic macrolides or derivatives, variants, mutants or homologues thereof wherein expression of said macrolide is indicative of the propensity of said potato to induce, up-regulate or otherwise contribute to the onset and/or progression of diabetes.

In another preferred embodiment, there is provided a method for assessing the propensity of a carrot to induce, up-regulate or otherwise contribute, subsequently to its consumption by a mammal, to the onset and/or progression of diabetes in said mammal, said method comprising screening said carrot for the expression of one or more β-cell toxic macrolides or derivatives, variants, mutants or homologues thereof wherein expression of said macrolide is indicative of the propensity of said potato to induce, up-regulate or otherwise contribute to the onset and/or progression of diabetes.

A “macrolide” is an antibiotic from the group of antibiotics produced by several strains of Streptomyces. These molecules exhibit a complex macrolytic structure and some are thought to inhibit protein synthesis by blocking the 50S ribosomal subunit while others inhibit vATPase activity. Macrolides are usually used clinically as broad spectrum antibiotics, particularly against gram positive bacteria, others are used as anti-nematode antibiotics. Reference to a “β-cell toxic macrolide” should be understood as a reference to a macrolide which either directly or indirectly adversely affects the functioning and/or morphology of the pancreatic islet β-cells. Within the context of the present invention, the β-cell is deemed to be adversely affected where at least one functional activity or morphological feature of the subject β-cell is modulated such that it is ablated, reduced or otherwise inappropriately altered. The adverse affect may be either permanent or transient. A direct effect is one where the macrolide acts on the β-cell itself to modulate one or more of its functional activities or morphological features while an indirect effect is one where the macrolide acts on a molecule other than a β-cell, which molecule in turn acts either directly or indirectly to adversely modulate one or more of the functional activities or morphological features of the βcell. It should be understood that this definition is inclusive in that the subject macrolide may exhibit one or more functional activities in addition to adversely modulating β-cell functioning and/or morphology. Preferably, the subject β-cell toxic macrolide is a bafilomycin or a concanamycin.

Accordingly, in one preferred embodiment there is provided a method for assessing the propensity of a plant or propagation material thereof to induce, up-regulate or otherwise contribute, subsequently to its consumption by a mammal, to the onset and/or progression of diabetes in said mammal, said method comprising screening said plant or propagation material thereof for the expression of one or more bafilomycin macrolides or derivatives, variants, mutants or homologues thereof wherein expression of said bafilomycin macrolide is indicative of the propensity of said plant or propagation material thereof to induce, up-regulate or otherwise contribute to the onset and/or progression of diabetes.

Preferably, said bafilomycin macrolide is one or more of bafilomycin A1, A2, B2 or C.

Even more preferably said plant or propagation material thereof is a tuberous vegetable and still more preferably a potato, beet or carrot.

In another preferred embodiment there is provided a method for assessing the propensity of a plant or propagation material thereof to induce, up-regulate or otherwise contribute, subsequently to its consumption by a mammal, to the onset and/or progression of diabetes in said mammal, said method comprising screening said plant or propagation material thereof for the expression of one or more concanamycin macrolides or derivatives, variants, mutants or homologues thereof wherein expression of said concanamycin A is indicative of the propensity of said plant or propagation material thereof to induce, up-regulate or otherwise contribute to the onset and/or progression of diabetes.

Preferably, said concanamycin is one or more of concanomycin A, B, C or D.

Preferably, said plant or propagation material thereof is a tuberous vegetable and still more preferably a potato, beet or carrot.

Without limiting the present invention to any one theory or mode of action, bafilomycin A1reduces vATPase activity in kidney cell homogenates. This effect persists for several days, indicative of a slow rate of clearance of the inhibitor activity from tissues. The vATPase inhibition that occurs in islet β cells results in altered insulin secretion, and would lead to poor glycaemic control in a subject animal. The effects include suppression of basal insulin secretion and changes in oral glucose tolerance by impairing the first phase of insulin secretion.

An indirect mechanism by which vATPase inhibition is thought to suppress basal insulin release is by the translocation of GLUT4 to the plasma membrane. Specifically, bafilomycin A1-induced GLUT4 translocation in vivo is thought to enhance the efficiency of insulin-independent glucose uptake by peripheral tissues resulting in a reduced insulin requirement and lower fasting plasma insulin levels (Chinni and Shishera, 1999). Thus the combined effect of GLUT4 translocation and suppression of basal insulin secretion by bafilomycin A1 is thought to lead to the reduced fasting plasma insulin levels. Coincident with the reduced fasting insulin levels is an increase in the levels of intra-islet immunoreactive insulin. Insulin biosynthesis and secretion are coordinated by the induction of insulin biosynthesis at the translational level by insulin secretagogues. As the fasting blood glucose concentrations remain normal, glucose stimulation of proinsulin biosynthesis will continue, which, in combination with suppressed insulin secretion, will result in accumulation of proinsulin/insulin within secretory vacuoles in the islets.

Glucose tolerance tests in mice revealed a higher and earlier peak in the blood glucose levels during oral glucose tolerance tests. The return to normal glucose levels 2 hours after glucose challenge, and the similarity in glucose-induced serum insulin levels in treated and untreated mice, suggests that the islet β-cells are still capable of glucose induced insulin secretion. This suggests that the altered glucose tolerance is due to a requirement for higher glucose levels to induce the release of sufficient insulin to stimulate glucose up-take. It is thought that bafilomycin A1interferes with secretory granule biogenesis at a stage prior to docking at the plasma membrane resulting in the deficiency in insulin secretion.

Still without limiting the present invention to any one theory or mode of action, it has also been surprisingly found that bafilomycin A1exposure affects islet β-cell morphology. The results of oral glucose tolerance tests on mice at 1 or 3 weeks after bafilomycin A1 exposure demonstrates a progressive increase in the blood glucose levels 15 minutes after glucose challenge. At the 3-week time point, islet morphology is normal, suggesting that the altered glucose tolerance is not due to a loss of β-cells, but rather to an intrinsic physiological defect. However, over the 90 day period, glucose tolerance, as assessed by random blood glucose levels, progressively worsens and the islets showed an altered morphology indicative of either fragmentation or neogenesis. β-cell mass is also appears to be reduced. This indicates that vATPase inhibition causes an islet β-cell functional defect that progressively worsens, eventually leading to β-cell death and/or neogenesis.

“Derivatives” of the macrolides defined herein include fragments, parts, portions and variants from natural or recombinant sources including fusion proteins. By “recombinant sources” is meant that the Streptomyces producing the subject macrolide have been genetically altered thereby resulting in modification to the macrolide expression product. This may occur for example, where a genetically modified organism is either intentionally or unintentionally released into the environment. Parts or fragments of the macrolide include, for example, active regions of the macrolide.

A “variant” or “mutant” of the subject macrolide should be understood to mean a macrolide which exhibits at least some of the functional activity of the macrolide of which it is a variant or mutant. The variation or mutation may take any form including a genetic or non-genetic variation or mutation. The subject variation or mutation may be naturally or non-naturally occurring.

By “homologue” is meant the macrolide, which is screened for in accordance with the method of the present invention, is derived from a species or genera other than that from which the macrolide is usually produced. This may occur, for example, where it is determined that a species of Streptomycete other than that which is currently known to produce the subject macrolide is nevertheless identified as producing a macrolide exhibiting similar functional characteristics or where it is determined that an Actinomycete genus other than Streptomyces, such as Microbispora, Micromonospora or Nocardiodes produces molecules which exhibit functional similarities to the macrolides defined herein.

The present invention is directed to screening plants, or propagation material thereof, for the propensity to contribute to diabetes onset and/or progression. By “propensity” is meant that a plant, if it expresses a β-cell toxic macrolide and is ingested, is more likely to contribute to the onset and/or progression of diabetes than a plant which does not express the macrolide. That is, it is an indication of relative risk. Accordingly, it should be understood that a plant which expresses a β-cell toxic macrolide may not contribute to diabetes in all individuals who ingest that plant. For example, there may be some individuals who, because of their genetic make up, are more predisposed to being adversely affected by a β-cell toxic macrolide than individuals of a different genetic make up. Since the full range of genetic factors contributing to diabetes predisposition are far from being understood, the method of the present invention is particularly useful for identifying dietary factors which will contribute to diabetes onset in at least some individuals, for example, those exhibiting a genetic susceptibility. Accordingly, it provides a system whereby an informed decision can be made in relation to diabetes risk factors associated with an individual's, or group of individuals', diets.

Reference to a β-cell toxic macrolide which “contributes” to the onset and/or progression of diabetes should be understood to mean either that the macrolide is the sole cause of the diabetes or that it is one of a number of contributing factors. For example, in some individuals the ingestion of a β-cell toxic macrolide may be the sole factor required to cause the onset of diabetes or to up-regulate, such as increasing severity of, an existing diabetes. Alternatively, ingestion of the β-cell toxic macrolide may be a factor which, together with the concurrent occurrence of other dietary or non-dietary factors (such as a genetic predisposition) causes the onset or up-regulation of diabetic conditions. In the absence of the macrolide, the consequent onset or up-regulation may not occur or may occur with significantly less severity. In the epidemiological sense, the macrolide exposure would be a component cause of an ensuing diabetes.

Accordingly, reference to “inducing, up-regulating or otherwise contributing” to the onset and/or progression of diabetes in a mammal should be understood as a reference to the induction, up-regulation or other contribution to any one or more symptoms of diabetes. Symptoms of diabetes include, but are not limited to, abnormal glucose levels or glucose level regulation, abnormal insulin levels, thirst, frequent urination, weight loss, blurred vision, headache and abdominal pain. It should be understood that the method of the present invention is directed to identifying the propensity of a plant or propagation material thereof to induce, up-regulate or otherwise contribute to the onset and/or progression of any one or more of these symptoms. By “progression” is meant up-regulation of the severity of the diabetes or the induction of a specific symptom which has either been in a remissive state or has not been experienced by the individual despite the individual's status as a diabetic. It also include the ongoing presence of the symptom where the subject symptom may otherwise have been reduced in severity or even disappeared.

Reference to “diabetes” should be understood as a reference to a condition in which insufficient levels of insulin are produced to maintain biologically normal glucose levels. As detailed hereinbefore, the diabetes which is the subject of the present invention may either be induced solely by the β-cell toxic macrolide or it may be induced by the subject β-cell toxic macrolide when acting in concert together with a number of other contributory factors or component causes. Alternatively, the β-cell toxic macrolide may act either alone or together with other factors to contribute to the progression of an existing diabetic condition. Accordingly, to the extent that the diabetes is a result of a number of contributing factors, the factors other than the β-cell toxic macrolide may include congenital defects in the pancreatic islet cells, the onset of an autoimmune response directed to the pancreatic β-cells (for example Type 1 diabetes/IDDM, slowly progressive adult onset IDDM which is also referred to as latent autoimmune diabetes in adults or LADA), defects in the functioning of the pancreatic islet cells caused by dietary factors (other than macrolides) or stress (for example Type 2 diabetes/adult onset diabetes non-insulin dependent diabetes mellitus, NIDDM), damage to the pancreatic islet cells such as, but not limited to, that caused by physical injury, the degeneration of pancreatic islet cells due to any one of a number of non-autoimmune conditions or as a side-effect to the onset or treatment of an unrelated disease condition. Accordingly, “diabetes” as referred to herein includes both typical Type I diabetes, Type 2 diabetes and other diabetic conditions including gestational diabetes.

The term “mammal” as used herein includes humans, primates, livestock animals (eg. horses, cattle, sheep, pigs, donkeys), laboratory test animals (eg. mice, rats, rabbits, guinea pigs), companion animals (eg. dogs, cats) and captive wild animals (eg. kangaroos, deer and foxes). Preferably the mammal is a human or a laboratory test animal. Even more preferably, the mammal is a human.

Suitable methods of screening plants, or propagation material thereof, for their expression of a β-cell toxic macrolide would be well known to the person of skill in the art and includes, but is not limited to:
(i) plant section staining or cell suspension analysis utilising technology directed to labelling the subject macrolide and detecting same.
For example, the target macrolide may be exposed to a specific antibody which may or may not be labelled with a reporter molecule. Depending on the amount of target and the strength of the reporter molecule signal, a bound target may be detectable by direct labelling with an antibody. Alternatively, a second labelled antibody, specific to the first antibody is exposed to the target-first antibody complex to form a target-first antibody-second antibody tertiary complex. The complex is detected by the signal emitted by the reporter molecule.
By “reporter molecule” as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (ie. radioisotopes) and chemiluminescent molecules.
In the case of an enzyme immunoassay (EIA), an enzyme is conjugated to the second antibody, generally by means of glutaraldehyde or periodate. As will be readily recognised, however, a wide variety of different conjugation techniques exist, which are readily available to the skilled artisan. Commonly used enzymes include horseradish peroxidase, glucose oxidase, beta-galactosidase and alkaline phosphatase, amongst others. The substrates to be used with the specific enzymes are generally chosen for the production, upon hydrolysis by the corresponding enzyme, of a detectable colour change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody hapten complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of hapten which was present in the sample.

Alternatively, fluorescent compounds, such as fluorescein and rhodamine, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic colour visually detectable with a light microscope. As in the EIA, the fluorescent labelled antibody is allowed to bind to the first antibody-hapten complex. After washing off the unbound reagent, the remaining tertiary complex is then exposed to the light of the appropriate wavelength the fluorescence observed indicates the presence of the hapten of interest. Inmunofluorescence and EIA techniques are both very well established in the art and are particularly preferred for the present method. However, other reporter molecules, such as radioisotope, chemiluminescent or bioluminescent molecules, may also be employed.
(ii) functional analyses based on screening for inhibition of vATPase activity in membrane fractions from cultured cells, organs from mammals, yeast or any other source of vATPase. vATPase activity is that proportion of total ATPase activity as measured by depletion of ATP or release of inorganic phosphate that is sensitive to bafilomycin or any other vATPase inhibitor.
(iii) identification of the macrolide via HPLC, gas chromatography, mass spectrometry, using appropriate standards.
For example, one approach for identifying bioactive secondary metabolites (such as bafilomycins and concanamycins) can be achieved utilising organic solvent extraction of samples, RP-HPLC separation of components in the extracts, testing of peak fraction in a bioassay and the identification of the active species by UV spectrometry, mass spectrometry and/or nuclear magnetic resonance. Without limiting the present invention to any one theory or mode of action, nanogram quantities of vATPase inhibitors and ionophores can be readily detected utilising the fact that the vATPase acidifies intracellular compartments by pumping protons across membranes. The ensuing pH gradient can be detected microscopically by the accumulation of acidotropic cell permeant dyes, such as acridin orange. When these pH gradients are ablated by ionophores (eg. nigericin), which facilitates the movement of ions across membranes, or by direct inhibition of the vATPase (ie. bafilomycins and concanamycins), the dye does not accumulate in intracellular compartments.

In addition to screening plant material for the expression of macrolides, and therefore determining the propensity of that plant to contribute to diabetes onset and/or progression, the identification of a causal link between plant expression of macrolides and diabetes onset has also facilitated the development of screening methodology directed to assessing the presence of macrolides in a mammal as an indicator either of that mammal's predisposition to developing diabetes or as a contributing factor to the existence and/or progression of an existing diabetic condition.

Accordingly, in another aspect there is provided a method of diagnosing the existence of a diabetes risk factor in a mammal, said method comprising screening said mammal for the expression of one or more β-cell toxic macrolides or derivative, variant, mutant or homologue thereof wherein the expression of said macrolide may be indicative of the risk of onset and/or progression of diabetes in said mammal.

Reference to “expression” should be understood as referring to the presence of the subject macrolide in the mammal. The presence of the macrolide may be due to ingestion of the macrolide via consumption of a contaminated plant, such as a tuberous vegetable, or it may be the result of consumption of the Streptomyces infected plant, which Streptomyces continue to produce the macrolide following their ingestion.

By “diagnosing” the existence of a “diabetes risk factor” is meant that the disclosed screening methodology enables one to determine either:
(i) that an individual who does not yet exhibit any diabetes related symptoms is at risk of having developed a predisposition to the development of diabetes due to ingestion of a β-cell toxic macrolide.
(ii) that the onset and/or progression of diabetes in a diabetic individual may have been induced or up-regulated by ingestion of a β-cell toxic macrolide.

Reference to “diabetes”, “mammal”, “β-cell toxic macrolide”, “derivatives, variants, mutants or homologues thereof” and “onset and/or progression” should be understood to have the same meanings as hereinbefore defined.

In a preferred embodiment there is provided a method of diagnosing the existence of a diabetes risk factor in a human said method comprising screening said human for the expression of one or more bafilomycin macrolides or derivative, variant, mutant or homologue thereof wherein the expression of said bafilomycin may be indicative of the risk of onset and/or progression of diabetes in said human.

Preferably said bafilomycin is bafilomycin A1, A2, B1, B2 and/or C.

In another preferred embodiment there is provided a method of diagnosing the existence of a diabetes risk factor in a human said method comprising screening said human for the expression of one or more concanamycin macrolides or derivative, variant, mutant or homologue thereof wherein the expression of said concanamycin A may be indicative of the risk of onset and/or progression of diabetes in said human.

Preferably said concanomycin is concanamycin A, B, C and/or D.

The screening methodology disclosed above should be understood to include both one off measurements of β-cell toxic macrolide levels in a plant, propagation material thereof, or in a mammal and multiple measurements conducted over a period of time (for example as may be required for the ongoing monitoring of plant infestation or an individual mammal's diabetes risk factor status or effectiveness of therapeutic or prophylactic protocols).

Another aspect of the present invention provides a diagnostic kit for assaying plants, propagation material thereof, or a biological sample comprising in a compartmental form a first compartment adapted to contain an agent for detecting a β-cell toxic macrolide and a second compartment adapted to contain reagents useful for facilitating the detection by the agent in the first compartment. Further compartments may also be included, for example, to receive a biological sample. The agent may be an antibody or other suitable detection molecule.

The elucidation of a correlation between diabetes onset and/or progression and the ingestion of β-cell toxic macrolides has now facilitated the development of prophylactic and therapeutic protocols directed to reducing, ameliorating or otherwise preventing diabetes in a mammal based on reducing levels of β-cell toxic macrolides such as via down-regulation of the functional activity of the macrolide or modulation of dietary intake to minimise ingested macrolide load.

Accordingly, another aspect of the present invention is directed to a method of preventing, reducing or otherwise ameliorating diabetes in a mammal, said method comprising down-regulating the functional activity β-cell toxic macrolides or derivatives, variants, mutants or homologues thereof, expressed by said mammal.

Preferably, said β-cell toxic macrolide is a bafilomycin or a concanamycin A and still more preferably said bafilomycin is bafilomycin A1, A2, B1, B2 and/or C3 and said concanamycin is concanamycin A, B, C and/or D.

Reference to “preventing, reducing or otherwise ameliorating” diabetes in a subject should be understood as a reference to the prevention, reduction or amelioration of any one or more symptoms of diabetes. As hereinbefore detailed, symptoms of diabetes include, but are not limited to, abnormal glucose levels or glucose level regulation, abnormal insulin levels, thirst, frequent urination, weight loss, blurred vision, headache and abdominal pain. It should be understood that the method of the present invention may either reduce the severity of any one or more symptoms or eliminate the existence of any one or more symptoms. Although complete normalisation is the most desirable, partial normalisation is nevertheless useful, for example, to reduce the risk of a Type 1 diabetic individual succumbing to a diabetic coma or to reduce reliance of intravenous insulin administration. The method of the present invention extends to preventing the onset of any one or more symptoms of diabetes. For example, in individuals who are predisposed to the development of diabetes (for example genetic predisposition), whose pancreatic islet cells are gradually degenerating or have suffered acute and irreparable injury to pancreatic islet cells, the method of the present invention may be employed to minimise any further degeneration to pancreatic β-cells.

Down-regulation of β-cell toxic macrolide functional activity may be achieved by one of several techniques, including but in no way limited to introducing into said mammal a proteinaceous or non-proteinaceous molecule which antagonises the β-cell toxic macrolide such as by neutralising the macrolide or at least partially down-regulating its functioning. In another example the subject proteinaceous or non-proteinaceous molecule may act on the Streptomyces to prevent macrolide expression and secretion. In still another example, the subject proteinaceous or non-proteinaceous molecule may act to inhibit or at least minimise the functional actions of the macrolide by acting on the β cell to protect it from the macrolide functional activity. That is, the molecule exhibits protective effects on the islet β cell and, more particularly, on adverse effects mediated by the toxic macrolides. These proteinaceous and non-proteinaceous molecules are hereinafter referred to as “the β-cell toxic macrolide antagonists”.

Said proteinaceous molecule may be derived from natural or recombinant sources including fusion proteins or following, for example, natural product screening. Said non-proteinaceous molecule may be, for example, a nucleic acid molecule or may be derived from natural sources, such as for example natural product screening or may be chemically synthesised. The present invention contemplates chemical analogs of β-cell toxic macrolide capable of acting as antagonists. Antagonists may be any compound capable of blocking, inhibiting or otherwise preventing β-cell toxic macrolide from carrying out its normal biological functions. Antagonists include monoclonal antibodies specific for β-cell toxic macrolide, or parts of β-cell toxic macrolide, and antisense nucleic acids which prevent transcription or translation of genes or mRNA involved in the synthesis of β-cell toxic macrolides.

For example the present invention may utilise antibodies to β-cell toxic macrolides including catalytic antibodies. Such antibodies may be monoclonal or polyclonal and may be selected from naturally occurring antibodies to β-cell toxic macrolide or may be specifically raised to a β-cell toxic macrolide. In the case of the latter, the β-cell toxic macrolide may first need to be associated with a carrier molecule. Alternatively, fragments of antibodies may be used such as Fab fragments. Furthermore, the present invention extends to recombinant and synthetic antibodies and to antibody hybrids. A “synthetic antibody” is considered herein to include fragments and hybrids of antibodies. The antibodies of this aspect of the present invention are particularly useful for immunotherapy and may also be used as a diagnostic tool for assessing apoptosis or monitoring the program of a therapeutic regimen.

For example, β-cell toxic macrolide can be used to screen for naturally occurring antibodies to β-cell toxic macrolide.

Said proteinaceous or non-proteinaceous molecule may act either directly or indirectly to modulate the expression of β-cell toxic macrolide or the activity of β-cell toxic macrolide. Said molecule acts directly if it associates with β-cell toxic macrolide to modulate the expression or activity of β-cell toxic macrolide. Said molecule acts indirectly if it associates with a molecule other than β-cell toxic macrolide which other molecule either directly or indirectly modulates the expression or activity of β-cell toxic macrolide. Accordingly, the method of the present invention encompasses the regulation of β-cell toxic macrolide expression or activity via the induction of a cascade of regulatory steps which lead to the regulation of β-cell toxic macrolide expression or activity.

Administration of the β-cell toxic macrolide antagonist in the form of a pharmaceutical composition, may be performed by any convenient means. The β-cell toxic macrolide antagonist or agent of the pharmaceutical composition is contemplated to exhibit therapeutic activity when administered in an amount which depends on the particular case. The variation depends, for example, on the human or animal and the antagonist. A broad range of doses may be applicable. Dosage regimes may be adjusted to provide the optimum therapeutic response. For example, several divided doses may be administered daily, weekly, monthly or other suitable time intervals or the dose may be proportionally reduced as indicated by the exigencies of the situation. The antagonist may be administered in a convenient manner such as by the oral, intravenous (where water soluble), intranasal, intraperitoneal, intramuscular, subcutaneous, intradermal or suppository routes or implanting (e.g. using slow release molecules). With particular reference to use of proteinaceous antagonists, these peptides may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

Without limiting the present invention to any one theory or mode of action, bafilomycin A1 has been shown to inhibit the vATPase activity. Accordingly, it is desirable to reduce levels of the vATPase inhibitor, being the β-cell toxic macrolide, in order to at least partially restore some vATPase activity. Further, since β-cell morphology degeneration occurs over a period of time, ongoing monitoring of an individual to detect changes in β-cell toxic macrolide levels provides a means timing the commencement of therapeutic and/or prophylactic treatment in order to prevent or at least reduce the incidence of β-cell morphology damage.

In accordance with these methods, the antagonist defined in accordance with the present invention may be coadministered with one or more other compounds or molecules. By “coadministered” is meant simultaneous administration in the same formulation or in two different formulations via the same or different routes or sequential administration by the same or different routes. By “sequential” administration is meant a time difference of from seconds, minutes, hours or days between the administration of the two types of molecules, These molecules may be administered in any order.

In a related aspect of the present invention, the subject undergoing treatment or prophylaxis may be in a human or animal in need of therapeutic or prophylactic treatment. In this regard, reference herein to “treatment” and “prophylaxis” is to be considered in its broadest context. The term “treatment” does not necessarily imply that a mammal is treated until recovery. Similarly, “prophylaxis” does not necessary mean that the subject will not eventually contract a disease condition. Accordingly, treatment and prophylaxis including amelioration of the symptoms of a particular condition or preventing or otherwise reducing the risk of developing a particular condition. The term “prophylaxis” may be considered as reducing the severity of onset of a particular condition “Treatment” may also reduce the severity of an existing condition or the frequency of acute attacks.

In a preferred embodiment the subject of the treatment is a mammal and still more preferably a human although the present invention is exemplified utilising a murine model, this is not intended as a limitation on the application of the method of the present invention to other species, in particular, humans.

In yet another further aspect the present invention contemplates a pharmaceutical composition comprising a β-cell toxic macrolide antagonist together with one or more pharmaceutically acceptable carriers and/or diluents. The antagonist is referred to as the active ingredients.

The pharmaceutical forms suitable for injectable use include sterile aqueous solutions (where water soluble) and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as licithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thirmerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives,-a dye and flavouring such as cherry or orange flavour. Of course, any material used in preparing any dosage unit form should be pharmaceutically pure and substantially non-toxic in the amounts employed. In addition, the active compound may be incorporated into sustained-release preparations and formulations.

Pharmaceutically acceptable carriers and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail.

The principal active ingredient is compounded for convenient and effective administration in effective amounts with a suitable pharmaceutically acceptable carrier in dosage unit form as hereinbefore disclosed. A unit dosage form can, for example, contain the principal active compound in amounts ranging from 0.5 μg to about 2000 mg. Expressed in proportions, the active compound is generally present in from about 0.5 μg to about 2000 mg/ml of carrier. In the case of compositions containing supplementary active ingredients, the dosages are determined by reference to the usual dose and manner of administration of the said ingredients.

The pharmaceutical composition may also comprise genetic molecules such as a vector capable of transfecting target cells where the vector carries a nucleic acid molecule capable of modulating macrolide expression or activity. The vector may be, for example, a viral vector. The present invention should be understood to extend to the use of such vectors in gene therapy.

It should be understood that in addition to down-regulating the functional activity of β-cell toxic macrolides which have been ingested by a mammal, the present invention also extends to decreasing the functional activity of β-cell toxic macrolides prior to their ingestion by a mammal. For example, it may be desirable to implement the large scale treatment of plant materials, such as tuberous vegetables, which are thought to be infected with bacteria that produce these macrolides. Such treatment could be performed, for example, prior to purchase of the vegetables by the consumer. Methods of treating the subject vegetables utilising one or more of the antagonistic agents described therein would be well known to those of skill in the art.

In yet another aspect there is provided a method of preventing, reducing or otherwise ameliorating diabetes in a mammal, said method comprising reducing consumption by said mammal of plants, or propagation material thereof, which plants express one or more β-cell toxic macrolides or derivatives, variants, mutants or homologues thereof.

Preferably, said β-cell toxic macrolide is a bafilomycin or a concanamycin and still more preferably said bafilomycin is bafilomycin A1, A2, B1, B2 and/or C and said concanamycin is concanamycin A, B, C and/or D.

In yet another most preferred embodiment said plant is a tuberous vegetable plant and still more particularly a potato, beet or carrot.

Further features of the present invention are described in the following non-limiting Examples.

### EXAMPLE 1

### Vacuolar ATPase Inhibitor, Bafilomycin A1, Induces Defective Insulin Secretion in Mice

(i) Materials and Methods

Mice

Male Balb/c mice were provided by the Monash University Central Animal House and Breeding Facility. The Monash University Department of Biochemistry Animal Ethics Committee approved all animal experimentation. Bafilomycin A1 (Sigma) was dissolved in 70% ethanol then diluted 1 in 10 with sterile phosphate buffered saline (PBS) to give a working stock of 4.8 μg/ml. Groups of between 3 and 9 mice as indicated were administered 12 μg per kilogram (body weight) bafilomycin A1 by intraperitoneal injection, which should achieve a final concentration in the blood of 275 nM bafilomycin A1 assuming a blood volume of 70 ml per kg for mice. This concentration is sufficient to inhibit in vitro vATPase activity with no detectable inhibition of either P-ATPase or F-ATPase activity (Drose et al., 1997). Control mice received the ethanol/PBS carrier solution that lacked the inhibitor.

V-ATPase Activity Assays

Seven control mice, 4 mice treated 1 day previously and 4 mice treated 7 days previously with bafilomycin A1 were anaesthetised with 35 mg per kg body weight pentobarbitone (Nembutal) and the kidneys removed and immediately frozen on dry ice and stored at −80° C. until processed. The kidneys were weighed then homogenised in liquid nitrogen using a mortar and pestle. 1 ml of 10 mM Tris-Cl pH 8.0 and 2 mM Mg₂Cl containing protease inhibitors was then added and the tissue was further homogenised with 5 strokes of a dounce homogeniser. One ml of 0.5 M sucrose, 40 mM Tris-Cl pH 8, 2 mM Mg₂Cl was then added to the homogenate and the suspension was clarified by centrifugation at 700×g for 10 minutes at 4° C. Supernatents containing soluble and membrane bound components were then assayed in duplicate for ATPase activity by incubating 100 μl of a 1:100 dilution of the tissue extract with 1 mM ATP in Tris buffered saline (TBS) for 30 minutes in the well of a microtitre plate in the presence or absence of 15 μl of a 1 μM solution of bafilomycin A1 in a final reaction volume of 200 μl. The inorganic phosphate released was measured using the one-step method of Chan et al., (1986), and absorbances were read in a microtitre plate reader at a wavelength of 660 nm. Results were corrected for background phosphate concentration and ATP autohydrolysis. The protein content of each kidney extract was determined using the Bradford reagent (Biorad).

Oral Glucose Tolerance Tests (OGTTS)

Groups of 6 control and 6 treated mice were fasted for 6-7 hours then anaesthetised by intraperitoneal injection of 35 mg/kg body weight of pentobarbitone (Nembutal) in order to minimise stress-induced variations in blood glucose. D-glucose (2 mg/g body weight) was delivered into the stomach by gavage as a 200 mg/ml solution. Blood glucose concentrations were determined for the fasted mice and at 15, 30, 60 and 120 minutes after glucose challenge as follows. Whole blood (approximately 60 μl) was collected from the tail vein and glucose concentrations were immediately determined by the glucose oxidase method using a YSI glucose analyser.

Measurement of Immunoreactive Insulin

Groups of 9 mice were treated with bafilomycin A1 or carrier solution then fasted, anaesthetised with penotobarbitone and challenged with glucose as described for the OGTT. Blood samples for insulin assay were collected from the orbital plexus from fasted mice or from mice 15 minutes after glucose challenge using 4×75 μl haematocrit tubes for each bleed. Serum samples were diluted 1 in 5 or 1 in 20 then assayed in duplicate for insulin content using a Linco sensitive rat insulin radioimmunoassay kit. Pancreata from the mice that were bled 15 minutes after glucose challenge were dissected and frozen on dry ice. The frozen pancreata were homogenised in 2 ml of PBS containing 1 mg/ml BSA per gram of pancreas by 10 strokes in a dounce homogeniser. The slurry was sonicated using 3×10 second bursts and then centrifuged at 12,000×g for 60 minutes. The supernatants were stored at −20° C. Insulin content was determined by sensitive rat insulin RIA using a 1 in 1,000 dilution of the homogenates.

Histology And Immunohistochemistry

The pancreas, liver, kidney and brain were dissected from anaesthetised mice and immediately fixed in formalin. Paraffin embedded sections (4 μm) were either stained with haemotoxylin and eosin or prepared for immunohistochemistry or indirect immunofluorescence. Immunostaining was performed on de-paraffinised sections as follows. The section was blocked in PBS containing 1% skim milk powder for 1 hour. Anti-insulin antiserum raised in Guinea pig (Dako) was diluted 1 in 50 in PBS 1% skim milk powder and left at room temperature for 60 minutes. Sections were washed in PBS and incubated with 1:200 dilution of horse radish peroxidase conjugated anti-guinea pig antiserum (Sigma) or a 1:400 dilution of FITC-conjugated anti-guinea pig serum (Sigma) for 1 hour. Endogenous peroxidase activity was inactivated using 0.5% hydrogen peroxide in methanol prior to the antibody incubations and bound HRP conjugated antibody was detected using Sigma FAST-DAB peroxidase tablets. FITC was detected using an Olympus epifluorescence microsocope and images were captured and analysed using a digital camera and MCID software.

Statistics

Data were analysed by ANOVA and Students t-tests where significant differences were indicated. Where the data were not normally distributed, Mann-Whitney U-test was used. Students t-tests were performed using Microsoft excel software. ANOVA and Mann-Whitney U-tests were performed using Statistica for Windows (Statsoft).

(ii) Results

Inhibition in vivo of vATPase activity after intraperitoneal administration to mice was ascertained by measuring bafilomycin A1 sensitive ATPase activity in kidneys, which are a rich source of vATPase enzyme. Assays were performed on homogenates of kidneys from 7 control mice or groups of 4 mice injected with bafilomycin A1 either 1 day or seven days previously (table 1). There was a 50% reduction of bafilomycin A1 sensitive ATPase activity at 1 day and 7 days after treatment (p<0.05, Students t-test) but not in bafilomycin A1 insensitive ATPase activity, indicating specific vATPase inhibition in the treated mice. There were no significant differences in the weight or protein contents of the kidneys of treated or control mice (table 1).

TABLE 1

| | Nmol/min/mg | | |
|---|---|---|---|
| | protein Control | 1 day | 7 day |
| total ATPase activity ± SD | 5.8 ± 1.5 | 4.4 ± 0.5 | 4.8 ± 0.8 |
| Bafilomycin A1 sensitive | 1.6 ± 0.6 | 0.80 ± 0.30 | 0.80 ± 0.30 |
| ATPase activity ± SD | | | |
| kidney % of animal | 0.80 ± 0.10 | 0.90 ± 0.11 | 0.90 ± 0.15 |
| weight ± SD | | | |

Oral Glucose Tolerance

The consequences of vATPase inhibition in vivo for islet β-cell function were determined by OGTT and measuring levels of blood glucose and plasma insulin. OGTTs were performed on the same groups of 6 mice at 2 hours, 7 days or 21 days after administration of either bafilomycin A1 or the carrier solution. Since there were no significant differences in the blood glucose concentrations between the 3 OGTT determinations for the control group these were combined for comparison with each of the OGTT determinations on the treated mice (FIG. 1). Two hours after administration of bafilomycin A1, the OGTT profile was similar to the controls (data not shown). Seven days after administration, the fasting and peak glucose concentrations were similar to controls, but bafilomycin A1 treated mice had significantly lower levels after 2 hours (4.1±0.4 vs 5.4±1.6 mM, p=0.006, Student's t-test). This effect was also apparent 21 days after treatment (4.2±0.2 mM versus 5.4±1.6 mM for controls p=0.01, Students t-test). Twenty one days after inhibitor administration, the peak glucose level attained by the treated mice was significantly higher than for the controls (12.5±2.4 mM for treated mice versus 9.5±1.8 mM for controls 15 minutes after glucose challenge, p=0.04, Students t-test). The fasting glucose levels of the treated mice showed no change.

Effect of Bafilomycin A1 on Plasma and Pancreatic Insulin

Plasma immunoreactive insulin levels were measured in fasting mice and at 15 minutes after glucose challenge in 2 groups of 8 mice at 7 days after treatment with either bafilomycin A1 or carrier solution. Bafilomycin A1 treated mice had a significantly lower fasting level of insulin than controls (1.2±0.8 versus 3.1±1.3, p=0.004, Students t-test). Fifteen minutes after glucose challenge the plasma insulin levels were similar (15±10 versus 13±11, p=0.8, Students t-test).

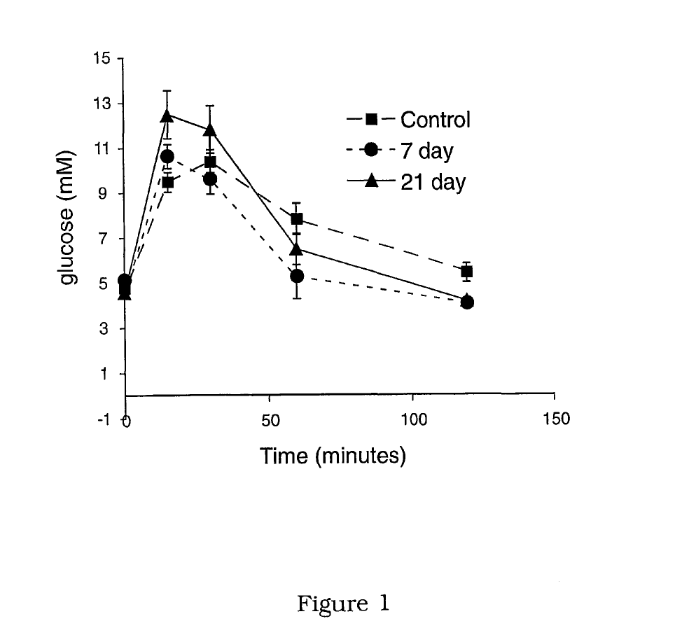

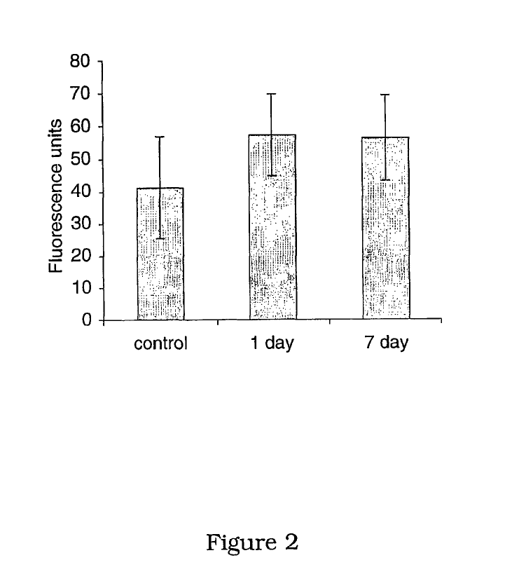

The total level of pancreatic insulin was determined by a semi-quantitative fluorescence method of detection (FIG. 2). Indirect immunofluorescence using an anti-insulin antibody was performed on paraffin embedded sections of pancreas, and the intensity of the fluorescence emitted by individual islets was quantified by digital image capture and densitometry using MCID software. Seventy islets were scored from 5 control mice, 45 islets from 3 mice treated with bafilomycin A1 1 day prior to pancreas dissection, and 49 islets from 3 mice treated with bafilomycin A1 7 days prior to pancreas dissection. The background fluorescence from exocrine pancreas adjacent to each islet was substracted before the data were analysed. The net fluorescence emitted by islets from control mice (median=41.5, range 7.7-72.7) was significantly lower than from mice treated with bafilomycin A1. This increase was significant at both 1 day (median=57.9, range 28.6-89.1) and 7 days (median=59.9, range 29.5-79.7) after bafilomycin A1 administration (p<0.0001 versus controls, Mann-Whitney U-test).

Pancreatic immunoreactive insulin content was determined directly by RIA on homogenates of pancreas. Mice treated 14 days previously were fasted and then challenged with glucose 15 minutes prior to removal of the pancreas. Pancreata from 5 treated mice contained 70% more immunoreactive insulin than pancreata from 4 controls (median=565, range 287-718 versus median=273, range 159-453 μg IRI per gram of pancreas, p<0.03, Mann-Whitney U-test).

Long-Term Effects of Bafilomycin A1 Treatment

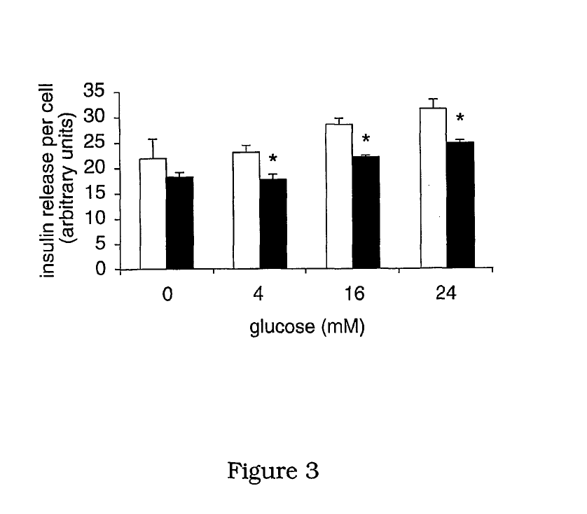

The long-term effects of bafilomycin A1 on blood glucose concentrations and islet morphology were investigated in 4 mice treated twice with a weekly interval. Random blood glucose levels (RBG) and weight were determined, weekly over 90 days and results were compared to a control group of 3 mice treated with carrier solution. Weekly measurement of blood glucose levels for 90 days after treatment revealed a small but significant increase in the blood glucose levels of treated mice over controls (FIG. 3, p<0.05, repeated measures ANOVA). The weight of the treated and control groups did not differ significantly.

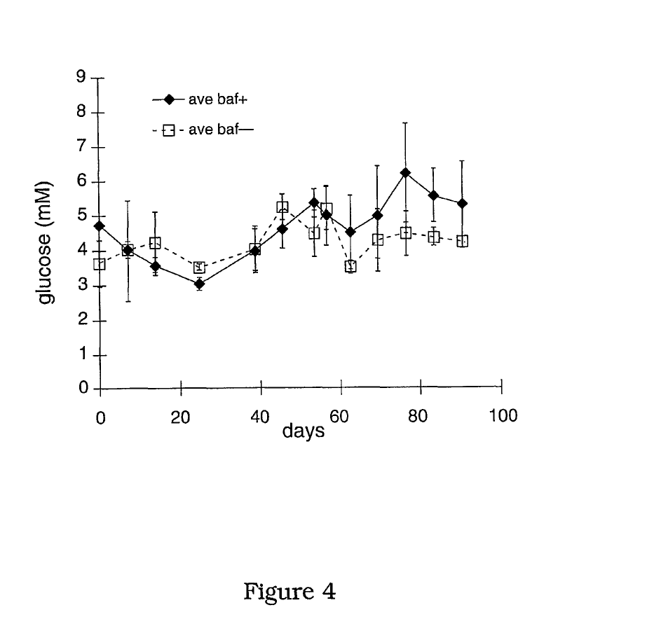

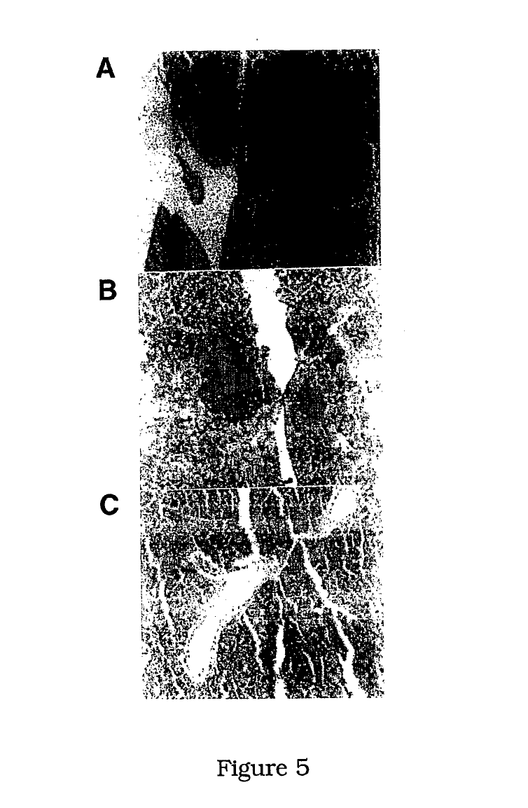

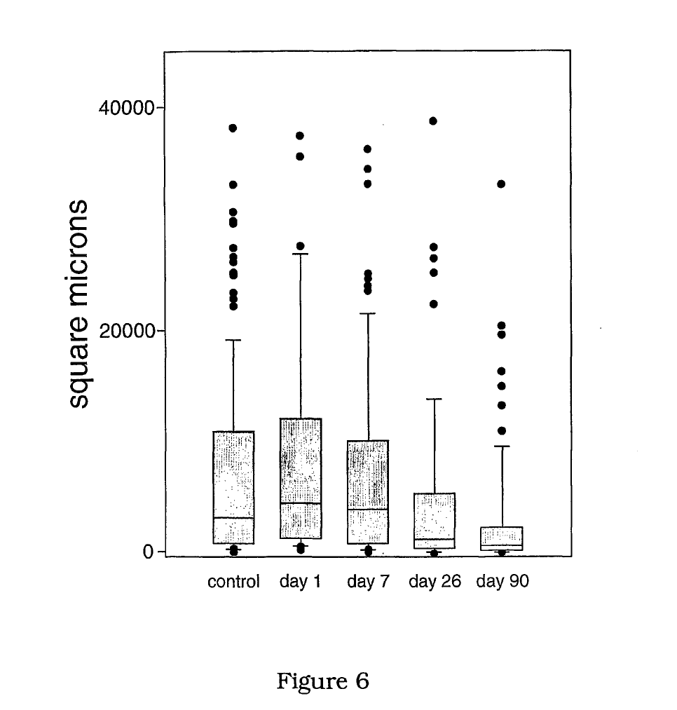

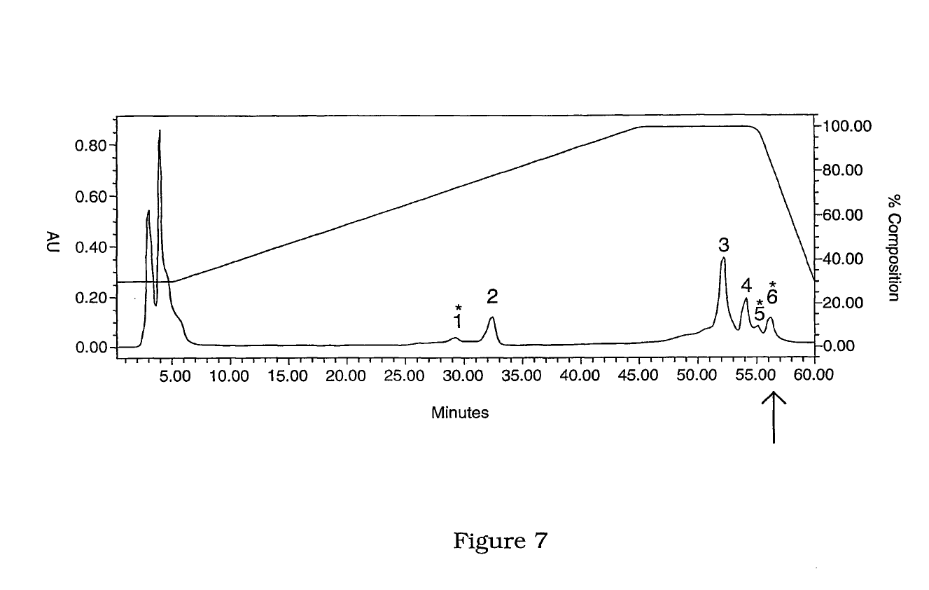

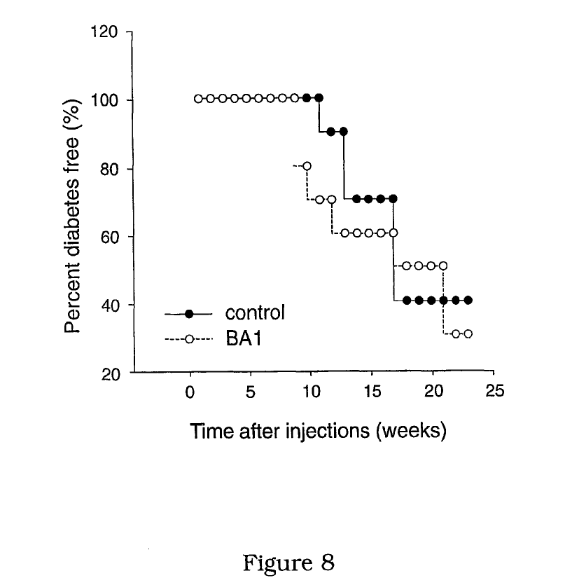

At the 90 day experimental end-point, the morphology of the islets was examined and found to show signs of fragmentation or neogenesis (FIG. 4). The size of the islets was measured and compared to those from the control mice and mice that were treated either 1 day, 7 days or 26 days previously (FIG. 6). There were no differences in islet size between the controls and 1 day or 7 day treatment groups but there was a significant decrease in islet size in mice treated 26 days or 90 days previously (p<0.001, Mann-Whitney U test). Examination of kidney, liver, testes, and brain did not reveal any gross morphological changes in these organs. The total islet area as a percentage of the total area of pancreas in the histological sections was 0.32% for controls, 0.33% for 1 day, 0.30% for 7 day treated mice, 0.23% for 26 day treated mice and 0.16% for 90 day treated mice but this difference was not significant (p=0.08, Mann-Whitney U test). For 223 islets from 12 control mice the mean islet size was 7900 μm², for 90 islets from 4 mice treated 26 days previously it was 4900 μm2, and for 81 islets from 4 mice treated 90 days previously and that received 2 doses of bafA1 it was 2800 μm².

### EXAMPLE 3

### Effect of Oral Administration of Bafilomycin A1 and Concanamycin A on β-Cells in Mice

The effect of oral administration of Bafilomycin A1 (BA1) and concanamycin A on β-cells in vivo could be tested. Three strains of mice that vary widely in their immunologic and metabolic characteristics, C57B1/6, SJL and BALB'c could be used to determine whether, like the repeated low doses of streptozotocin model, islet autoimmunity is induced by the treatment in mice with the appropriate genetic background. BA1 could be administered orally by gavage or by intraperitoneal injection on each of 5 consecutive days. Details of such treatments on each of the 3 strains are shown in table 2.

TABLE 2

| | | Dose of BA1 or | |
|---|---|---|---|
| | Administration | concanamycin (ng/gram | Number of |
| Group | route | body weight) | mice per group |
| 1 | oral (gavage) | 0 | 6 |
| 2 | | 12 | 6 |
| 3 | | 24 | 6 |
| 4 | IP injection | 12 | 6 |

Islet β-cell function could be assessed by an oral glucose tolerance test (OGTT) on day 6. The treatments are expected to suppress insulin secretion and lead to impaired glucose tolerance. Those strain/dose combinations that produce glucose intolerance could be examined further by measurement of levels of plasma and islet insulin. Pancreatic histology could be examined in 3 mice per group at 10 or 20 days after the last dose has been administered to test for deranged islet morphology and apoptosis using the TUNEL assay. These experiments could confirm and extend earlier observations that BA1 induces aberrant β-cell function in vivo and whether BA1 and concanamycin A are effective when administered orally.

### EXAMPLE 4

### Correlations Between Potato Consumption, Potato Scab Infestation and Type 1 Diabetes Incidence

The Tasmanian Insulin-Treated Diabetes Register represents a unique resource for the study of Type 1 diabetes epidemiology due to the structure and stability of the Tasmanian population. All Tasmanian residents who use insulin to treat their diabetes and are aged under 65 years are asked to register. The majority of registrants are recruited through the diabetes clinics around the state. Diabetes Educators provide a registration card to those eligible to complete and sign, then voluntary registrants forward these forms to the Menzies Centre. New registrants are also recruited through those already on the Register eg relatives, Diabetes Australia, media publicity, GPs and pharmacies, and in the past have been recruited through the National Diabetes Services Scheme. Some strengths of the Register include its completeness (84%), the rapport of the Menzies Centre staff with the clinicians involved and the co-operativity of the registrants.

Blood has been collected from 1,342 individuals on the Register for serological studies. Selection of Type 1 diabetes for this proposal is based on insulin treatment, original clinical presentation and medical history from treating physician. Auto-antibody profile & C-peptide, where available, are used as additional confirmatory information.

Environmental exposures relevant to Type 1 diabetes in the Tasmanian population could be examined through a case-control design based on an environmental questionnaire. Questions regarding the consumption of tuberous vegetables, particularly potatoes, as well as relevant confounders including environmental exposures previously associated with Type 1 diabetes, such as breast feeding (duration, exclusive and total), cow's milk intake (cow's milk based formula, cow's milk, dairy products), childhood viral infections and vaccinations could be measured.

At least 400 confirmed cases of Type 1 diabetes with complete clinical and phenotypic data are currently available and an equal number of controls from the electoral role, frequency matched by age and sex, are selected. Estimates of statistical power allowing for 5% false positives are as follows: detection with 80% power of: (a) odds-ratio (OR)=1.5-1.7 for enterovirus (18% controls affected), cow's milk (66% cases exposed) and no breast feeding (47% controls exposed).

It is recognised that a retrospective dietary questionnaire is prone to recall errors and so the hypothesis cannot be rejected based on a negative result. Consequently, other approaches can be used to seek correlations between exposure to Streptomyces infested tuberous vegetables and Type 1 diabetes incidence, including correlations between the annual incidence of Type 1 diabetes and the annual prevalence of potato scab in Tasmania

### EXAMPLE 5

### Streptomyces Toxins in Infected Vegetables

All plant pathogenic strains of Streptomyces sp. produce one or more related phytotoxins (thaxtomins) whilst non-pathogenic strains do not. These toxins are capable of inducing complete common scab disease symptoms when applied to developing tubers in absence of the pathogen itself. Production of other toxic secondary metabolites also appears to be a prevalent feature of plant-pathogenic Streptomyces. For example, the S. scabies type strain and another scab-causative species isolated in Japan produce concanamycins A and B and the bafilomycin and concanamycin -producing species S. griseus and S. diastatochromogenes are among the many species isolated from tuberous vegetables.

An approach for identifying bioactive secondary metabolites that inhibit important cellular processes in mammalian cells has been developed. This approach involves organic solvent extraction of samples, RP-HPLC separation of components in the extracts, testing of peak fractions in a bioassay, and identification of the active species by UV-spectrophotometry, mass spectrometry and/or nuclear magnetic resonance. Nanogram quantities of v-ATPase inhibitors and ionophores can be readily detected utilizing the fact that the vATPase acidifies intracellular compartments by pumping protons across membranes. The ensuing pH gradient can be detected microscopically by the accumulation of acidotropic cell permeant dyes, such as acridine orange. When these pH gradients are ablated by ionophores (i.e. nigericin), which facilitate the movement of ions across membranes, or by direct inhibition of the v-ATPase (i.e. bafilomycins and concanamycins), the dye does not accumulate in intracellular compartments.

Method

Samples of potato skin and flesh or broth cultures (2L oatmeal broth or Schnurer broth with vigorous agitation at 30° C. for 14 days) were ground and extracted using ethyl acetate or chloroform. Extracts were separated by RP-HPLC on a Deltapak C18 analytical column using a waters 600 solvent delivery system, 770 autosampler and 996 dual photodiode array detector using wavelengths of 210 and 254 nm. Peak fractions were collected and tested by bioassay as follows; COS7 cells were cultured in standard media containing a 1 in 20-25 dilution of the HPLC peak fraction for 1 hour. The media was removed and replaced with media containing 2 μg/ml acridine orange and incubated at 37 degrees Celsius/5% carbon dioxide in a humidified atmosphere for 15-20 mins. The cells were then examined by epifluorescence microscopy using blue excitation. Peak fractions that inhibited acridine orange uptake were further characterised by mass spectrometry using a Micromass ZMD ESI-MS equipped with a Gilson 306 HPLC 215 liquid handler and pumps and an Agilent 1100 series dual photodiode array detector.

Results

Extracts from 13 Australian Streptomyces strains isolated from infested vegetables have been analysed and 4 that produce toxins have been identified. An extract of skin from a common scab infected potato also abolished proton gradients in mammalian cells. Mass spectrometry revealed that one of these toxins was the previously characterized ionophore nigericin, while 3 others were vATPase inhibitors belonging to the bafilomycin and concanamycin classes of plecomacrolide antibiotics (Table 3).

TABLE 3
Plecomacrolide and ionophore toxins produced by scab-causative
Streptomyces. The toxins were identified on the basis of (i)
RP-HPLC retention time of unknowns compared to standards,
(ii) inhibition of acridine orange uptake in a bioassay, and (iii) Electron
Ionisation Mass Spectrometry (EI-MS) of bioactive fractions.

| Organism | Toxins |
|---|---|
| Streptomyces sp. EF-73 | Bafilomycins |
| Streptomyces hydroscopicus | Nigericin, Concanamycins |
| Streptomyces sp. EF-52 | Nigericin |
| Streptomyces sp. 80/2-1 | Nigericin, Bafilomycins |

An example chromatographic profile identifying bafilomycin in an extract of a Streptomyces isolated from a potato scab lesion is shown in FIG. 7.

### EXAMPLE 6

### V-ATPase Inhibitors Affect Pancreatic Islet Morphology.

The effect of vATPase inhibitors on the size and number of pancreatic islets was analysed in C57B1/6J using either bafilomycin A1 (BA1) or concanamycin A (CMA). The effect of Streptozotocin, an islet beta-cell specific toxin produced by Streptomyces achromogenes was also examined, to enable comparison with the effect of vATPase inhibitors.

Methods

Groups of 4 female C57B1/6J mice were injected with 5 doses on 5 consecutive days of 12 ng/g body weight BA1, CMA or the well characterised islet β-cell toxin streptozotocin. Pancreases were removed on day 84 and 3 formalin-fixed sections per mouse were immunostained for insulin. The area of insulin-positive islets was measured on digital images using MCID software.

Results

Intraperitoneal injection of 12 ng/gram body weight of BA1 or CMA induced marked changes in islet size. Both BA1 and CMA treatment increased the number of small islets while large islets were still present (Table 4). This contrasts with the effects of the β-cell toxin streptozotocin, which reduces the number of islets but does not induce the formation of new islets. These results demonstrate that the effects of vATPase inhibitors on islet size are not peculiar to the particular mouse strain or the sex of the mice, nor are they restricted to bafilomycin A1, since concanamycin A1 likewise altered islet morphology in C57B 1/6J mice despite differences in molecular structure.

TABLE 4
BA1 and CMA induce the formation of small islets

| | No. of | Median | P value vs control | No. islets per |
|---|---|---|---|---|
| | islets | size | (Mann-Whitney U- | mm² pancreas |
| Treatment | scored | (μm²) | test) | section |
| None | 188 | 5200 | NA | 0.65 |
| BA1 | 193 | 3830 | 0.004 | 0.85 |
| CMA | 145 | 3170 | <0.0001 | 0.78 |
| Streptozotocin | 106 | 5890 | 0.19 (ns) | 0.46 |

### EXAMPLE 7

### Acceleration of Diabetes Onset in Non-Obese Diabetic (Nod) Mice

The NOD mouse develops spontaneous diabetes due to an autoimmune mediated destruction of the pancreatic islet β-cells. The characteristics of diabetes development in the NOD mouse parallel those in humans, making the NOD mouse a useful model of human Type 1 diabetes. Lymphocytic infiltrates in the pancreatic islets, termed insulitis, first develops in NOD mice soon after weaning at 4 weeks of age and hyperglycaemia first appears from 18 to 32 weeks of age. Bafilomycin A1 was administered to NOD mice to test whether the onset of hyperglycaemia was accelerated.

Methods

Eleven-week-old female NOD/lt mice were injected intraperitoneally with either 12 ng per gram body weight of bafilomycin A1 (10 mice) or phosphate buffered saline carrier solution (10 mice) on each of 5 consecutive days. Blood glucose was monitored weekly and diabetes defined as a blood glucose measurement greater than 11 mmol/L.

Results

The first 3 NOD mice to develop hyperglycaemia were from the group treated with bafilomycin A1, indicating a slight acceleration of islet beta cell destruction and hyperglycaemia (FIG. 8)

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

Bibliography:

Amrani A, Durant S, Throsby M, Coulaud J, Dardenne M, Homo-Delarche F., Endocrinology., 139:1115-1124 (1998)
Arnush M, Heitmeier M R, Scarim A L, Marino M H, Manning P T, Corbett J A., Journal of Clinical Investigation., 102:516-526 (1998)
Chaillous L, Rohme V, Maugendre D, Lecomte P, Marechaud R, Marre M, et al., Metabolism: Clinical & Experimental., 45:306-314 (1996)
Chan K, Delfert D, Junger K D., Analytical Biochemistry., 157:375-380 (1986)
Chinni S R and Shisheva A., Biochemical Journal., 339:599-606 (1999)
Corbett J A, Wang J L, Sweetland M A, Lancaster J R, Jr., McDaniel M L., Journal of Clinical Investigation., 90:2384-2391 (1992)
Drose S, Altendorf K., Journal of Experimental Biology., 200:1-8 (1997)
Forgac M., Journal of Biological Chemistry., 274:12951-12954 (1999)
Forgac M., Journal of Biological Chemistry., 274:1301-1305 (1999)
Hostens K, Pavlovic D, Zambre Y, Ling Z, Van Schravendijk C, Eizirik D L, et al., Journal of Clinical Investigation., 104:67-72 (1999)
Kuliawat R, Arvan P., Journal of Cell Biology., 126:77-86 (1994)
Lindgren F A, Hartling S G, Dahlquist G G, Binder C, Efendic S, Persson B E., Diabetic Medicine., 8:638-643 (1991)
Orci L, Halban P, Perrelet A, Amherdt M, Ravazzola M, Anderson R G., Journal of Cell Biology., 126:1149-1156(1994)
Paquet L, Shou A, Chang E Y, Mains R E., Molecular & Cellular Endocrinology., 120:161-168 (1996)
Porte D., Annals of the New York Academy of Sciences., 892:73-8 (1999)
Rabinovitch A., Diabetes-Metabolism Reviews., 14:129-1561 (1998)
Roder M E, Knip M, Hartling S G, Karjalainen J, Akerblom H K, Binder C., The Childhood Diabetes in Finland Study Group. Journal of Clinical Endocrinology & Metabolism., 79:1570-1575 (1994)
Sjoholm A., Cell Death & Differentiation., 5:461-468 (1998)
Swallow C J, Grinstein S, Sudsbury R A, Rotstein O D., Journal of Experimental Medicine., 174:1009-1021 (1991)
Temple R C, Carrington C A, Luzio S D, Owens D R, Schneider A E, Sobey W J, et al., Lancet., 1:293-295 (1989)
Tojo A, Guzman N J, Gag L C, Tisher C C, Madsen K M., Americal Journal of Physiology., 267:F509-15 (1994)

1. A method for assessing the propensity of an ingestible agent to induce, up-regulate or otherwise contribute, subsequently to its ingestion by a mammal, to the onset, and/or progression of diabetes in said mammal, said method comprising screening said agent for the expression of one or more β-cell toxic macrolides or derivatives, variants, mutants or homologues thereof wherein expression of said macrolide is indicative of the propensity of said agent to induce, up-regulate or otherwise contribute to the onset and/or progression of diabetes.

2. The method according to claim 1 wherein said ingestible agent is a plant or propagation material thereof.

3. The method according to claim 2 wherein said plant is a tuberous vegetable.

4. The method according to claim 3 wherein said tuberous vegetable is a potato, beet or carrot.

5. The method according to any one of claims 1-4 wherein said β-cell toxic macrolide is bafilomycin or derivative, variant, mutant or homologue thereof.

6. The method according to claim 5 wherein said bafilomycin is bafilomycin A1, A2, B11, B2 and/or C.

7. The method according to claim 6 wherein said bafilomycin is bafilomycin A1.

8. The method according to any one of claims 1-4 wherein said β-cell toxic rnacrolide is a concanamycin or derivative, homologue, mutant or variant thereof.

9. The method according to claim 8 wherein said concanamycin is concanamycin A, B, C and/or D.

10. The method according to claim 9 wherein said concanamycin is concanamycin A.

11. A method of diagnosing the existence of a diabetes risk factor in a mammal, said method comprising screening said mammal for the expression of one or more β-cell toxic macrolides or derivatives, variants, mutants or homologues thereof wherein the expression of said macrolide may be indicative of the risk of the onset and/or progression of diabetes in said mammal.

12. The method according to claim 11 wherein said mammal is a human.

13. The method according to claim 11 or 12 wherein said β-cell toxic macrolide is a bafilomycin or derivative, homologue, mutant or variant thereof.

14. The method according to claim 13 wherein said bafilomycin is bafilomycin A1, A2, B1, B2 and/or C.

15. The method according to claim 14 wherein said bafilomycin is bafilomycin A1.

16. The method according to claim 11 or 12 wherein said β-cell toxic macrolide is a concanamycin or derivative, homologue, mutant or variant thereof.

17. The method according to claim 16 wherein said concanamycin is concanamycin A, B, C and/or D.

18. The method according to claim 17 wherein said concanamycin is concanamycin A.

19. A diagnostic kit for assaying plants, propagation material thereof, or a biological sample, said kit comprising in a compartmental form a first compartment adapted to contain an agent for detecting a β-cell toxic macrolide and a second compartment adapted to contain reagents useful for facilitating the detection by the agent in the first compartment.

20. The kit according to claim 19 wherein said plant is a tuberous vegetable.

21. The kit according to claim 20 wherein said tuberous vegetable is a potato, beet or carrot.

22. The kit according to any one of claims 19-21 wherein said β-cell toxic macrolide is a bafilomycin or derivative, variant, mutant or homologue thereof

23. The kit according to claim 22 wherein said bafilomycin is bafilomycin A1, A2, B 1, B2 and/or C.

24. The kit according to claim 23 wherein said bafilomycin is bafilomycin A1.

25. The kit according to any one of claims 19-21 wherein said β-cell toxic macrolide is a concanamycin or derivative, homologue, mutant or variant thereof.

26. The kit according to claim 25 wherein said concanamycin is concanamycin A, B, C and/or D.

27. The kit according to claim 26 wherein said concanamycin is concanamycin A.

28. A method of preventing, reducing or otherwise ameliorating diabetes in a mammal, said method comprising down-regulating the functional activity of β-cell toxic macrolides or derivatives, variants, mutants or homologues thereof, expressed by said mammal.

29. The method according to claim 28 wherein said mammal is a human.

30. The method according to claim 28 or 29 wherein said β-cell toxic macrolide is a bafilomycin or derivative, homologue, mutant or variant thereof.

31. The method according to claim 30 wherein said bafilomycin is bafilomycin A1, A2, B1, B2 and/or C.

32. The method according to claim 21 wherein said bafilomycin is bafilomycin A1.

33. The method according to claim 28 or 29 wherein said β-cell toxic macrolide is a concanamycin or derivative, homologue, mutant or variant thereof.

34. The method according to claim 33 wherein said concanamycin is concanamycin A, B, C and/or D.

35. The method according to claim 34 wherein said concanamycin is concanamycin C.

36. A pharmaceutical composition comprising a β-cell toxic macrolide antagonist together with one or more pharmaceutically acceptable carriers and/or diluents.

37. The pharmaceutical composition according to claim 36 when used in accordance with the method of any one of claims 28-33.

38. A method of preventing, reducing or otherwise ameliorating diabetes in a mammal, said method comprising reducing consumption by said mammal of plants, or propagation material thereof, which plants express one or more β-cell toxic macrolides or derivatives, variants, mutants or homologues thereof.

39. The method according to claim 38 wherein said ingestible agent is a plant or propagation material thereof.

40. The method according to claim 39 wherein said plant is a tuberous vegetable.

41. The method according to claim 40 wherein said tuberous vegetable is a potato, beet or carrot.

42. The method according to any one of claims 38-41 wherein said β-cell toxic macrolide is a bafilomycin or derivative, variant, mutant or homologue thereof.

43. The method according to claim 42 wherein said bafilomycin is bafilomycin A1, A2, B1, B2 and/or C.

44. The method according to claim 43 wherein said bafilomycin is bafilomycin A1.

45. The method according to any one of claims 38-41 wherein said β-cell toxic macrolide is a concanamycin or derivative, homologue, mutant or variant thereof.

46. The method according to claim 45 wherein said concanamycin is concanamycin A, B, C and/or D.

47. The method according to claim 46 wherein said concanamycin is concanamycin A.
